(19) United States
(12) Patent Application Publication
(10) Pub. No.: US 2026/0045355 A1
(43) Pub. Date: Feb. 12, 2026

(54) SYSTEMS AND METHODS FOR ANATOMY GUIDED IMAGE ANIMATION WORKFLOW

(71) Applicant: Aaron D. ODOM, Bradenton, FL (US)

(72) Inventor: Aaron D. ODOM, Bradenton, FL (US)

(21) Appl. No.: 19/294,866

(22) Filed: Aug. 8, 2025

Related U.S. Application Data
(60) Provisional application No. 63/681,838, filed on Aug. 11, 2024.

(51) Int. Cl. G16H 30/40; G06T 7/33; G16H 30/20

(52) U.S. Cl. CPC G16H 30/40; G06T 7/344; G16H 30/20

(57) ABSTRACT

Disclosed herein are systems and methods for medical imaging using anatomy guided image animation workflow. The system and method comprise applying auto-contouring to a medical image; assigning a deformation model to each structure; using a registration algorithm to perform registration; generating a common coordinate system; generating a first displacement map, where the first displacement map shows the displacement of structures from the target image set to the source; generating a first deformation map using the registration algorithm; applying the first deformation map to the source image set to align the geometry of the source to a target position; and generating a deformed, partially synthetic source image.

### CROSS-REFERENCE TO RELATED APPLICATIONS

This application claims benefit of priority under 35 U.S.C. § 119(e) of U.S. Ser. No. 63/681,838, filed 11 Aug. 2024, the entire contents of which is incorporated herein by reference in its entirety.

### BACKGROUND

### Field

Systems and methods for medical imaging using anatomy guided medical image animation workflow are described.

### Description of the Related Art

In some medical imaging workflows and imaging intensive medical treatment protocols, e.g. radiation therapy, differences in patient position between image sets can cause difficulty in correlating information from one set to another. Various imaging technologies have been developed to acquire internal images of the human anatomy, such as computed tomography (CT), magnetic resonance (MR), positron emission tomography (PET), and ultrasound (US). Often, the medical imaging field uses multiple imaging modalities to correlate anatomical information on one image set with information on another, e.g., the geometry, progression over time, metabolic status, etc. In some medical fields, such as radiation therapy, detailed information is required which cannot be measured in a single imaging modality. Thus, multiple imaging sessions are required often involving multiple imaging modalities. Information gathered via different imaging sessions or different imaging modalities must be correlated from one volumetric image set to another. Rigid registration tools were developed to establish a common coordinate system between image sets for the correlation and localization of information from one set to another. Differences in the patient's position between image sets, such as arms at side versus arms above the head, differences in anatomy such as weight gain or loss, or differences in the phase of the breathing cycle, can introduce ambiguity in the correlated anatomical information, which can exist in a part or the whole of an anatomical structure or can be movable inside the structure. This ambiguity can lead to errors which can include localization, measurement, or identification of the specific anatomical feature of interest.

Deformable registration tools have been developed to allow adjustment for changes in position but fail in areas of dramatic motion. Existing deformable registration tools have specifically performed poorly for structures whose boundaries are not easily visualized and whose motions depend largely on the position of surrounding structures or are otherwise difficult to define e.g., axial lymph nodes with respect to the position of the humerus or inguinal lymph nodes with respect to the femur position.

Deforming prior imaging to the treatment planning image set, or simulation CT, also lacks an independent validation. No mathematical routine exists to assess the validity or quantify the uncertainty of the process. The possibility of certain structures to change in size and shape over time and the existence of many small, somewhat ambiguous, somewhat mobile, soft-tissue structures which resemble one another, allows for the possibility of confusing one structure for another when using an algorithm that only examines patterns in pixel intensity. Using the simulation CT as the target for the deformation will necessarily propagate any error or subpar features of the simulation CT, and may, in fact, treat subpar features in the simulation CT as the definition of a good deformation result. When the deformation algorithm returns two image sets which have the pleasant appearance of congruent intensities, it is easy for the user to interpret this as a high-quality result, however a result that is pleasing in appearance is not evidence of its quality. An independent process, by which a prior image set is animated and repositioned, with built-in empirical and analytical uncertainties, and then subsequently compared to a simulation CT would allow for the quantification of uncertainty in the final product and would limit the unintentional masking of error.

The current deformation process enables a false confidence in the reproducibility of a given motion between two positions as both image sets are simple snapshots in time. Certain structures are mobile inside the human body apart from any skeletal manipulation or change in position or posture. The possibility exists that a patient's skeletal structure could be manipulated iteratively in the same position with certain structures being localized in different places each time. If the user becomes accustomed to the idea that a high-quality deformation results in a high level of congruence between all structures, or if one image set is used as the standard for the deformation process, the temporal uncertainty in the localization of the structure of interest is ignored.

Uniform standards have been developed for margins of uncertainty surrounding the structures of interest in radiation therapy treatment planning, however, these margins do not adequately consider asymmetry in the uncertainty of the localization or variance in the magnitude of uncertainty due to the quality of processes like deformation and registration.

In many current applications, e.g. radiation therapy, structures of interest may not be easily imaged, thus, surrogates are used. In the application of surface-guided-radiation-therapy (SGRT) for example or in 4DCT imaging of the breathing cycle, the patient surface or objects placed on or against the patient's surface are surrogates for the internal structures of interest because of the visibility or ease of imaging of the surrogate. In these applications, tolerances are defined which include certain small deviations in the treatment and exclude certain other deviations. The tolerances are often applied uniformly to the surrogate, and the magnitude of the constraints often relates to the acceptable motion of the structure of interest. The magnitude of motion of the surrogate which correlates to the acceptable motion of the target may or may not be defined. Thus, the potential exists for the surrogate tolerances to allow an arbitrary range of motion in the structure of interest.

Many patients in radiation oncology require multiple imaging sessions, and often multiple imaging modalities. Often patients are imaged for simulation using CT when a prior whole-body CT has been acquired either as a stand-alone imaging modality or in parallel with a PET scan. Current processes are not able to use this prior imaging due to the differences in the tabletop, the patient position, and the lack of devices which are necessary for the radiation therapy treatment.

Non-value-added time in the radiation therapy treatment planning workflow is an additional symptom of the above failures. In the treatment planning process, the patient is imaged in the presumed treatment position at the discretion of the physician and with the input of the technical team. While this is often established by the consensus of the team, it is not uncommon to find multiple positions which have potential advantages due to unknowns regarding the relative locations of the patient's internal anatomy in each potential target position. As the treatment plan generates dose distributions on the 3D simulation CT image set, a change in patient position necessitates the acquisition of a new image set, delaying the lengthy process, often by multiple days.

Dose-volume histograms (DVH) have long been a primary quality assurance tool in the radiation therapy treatment planning process. The DVH plots dose received along the horizontal axis and structure volume on the vertical axis. While the DVH remains an essential tool in radiation therapy treatment planning, DVH evaluation is somewhat subjective except for specific points which are routinely audited and is somewhat vague in terms of the specific shape and uniformity of dose distribution inside a structure or spilling out of the structure boundaries. To date, in many treatment planning applications it is not trivial to specify how a particular dose might be distributed in a given structure, and it is often time consuming when it is possible to do so. Such adjustments to the dose distribution are often done retroactively by generating dose distributions, performing an evaluation, and subsequently modifying the plan and dose distribution.

Consequently, there is a need for new medical imaging systems and methods that can improve accuracy of correlation, particularly in areas where anatomical motion can be dramatic, while maintaining effective computational efficiency. There is a need to develop an independent system of validation rather than setting one image as the standard for congruence. There is a need to develop a system for assessing spatial uncertainty which considers the correlation between anatomical surrogates and the structure of interest, as well as the quality of the processes of fusion, in addition to the traditional considerations.

There is also an opportunity to use prior imaging to the advantage of the patient and the treatment planning team, by incorporating artificial intelligence (AI) into a workflow which will leverage prior imaging data in order to proactively identify the ideal patient position, generate predictive dose distributions, facilitate the use of proactive graphical dose-volume constraints so that a desired dose is attached not just to a structure but to a specific location in or around the structure of interest, at the onset of the treatment planning process. Achieving these objectives at the beginning of the treatment planning process will remove non-value-added time which is inevitable with the current radiation therapy treatment planning methodology.

### SUMMARY

Provided herein are systems and methods for medical imaging that include: receiving one or more volumetric medical image sets, which can include, but is not limited to a computed tomography scan or a magnetic resonance image, where the medical image includes: one or more voxels, one or more pixels, or a combination thereof, where the medical image includes one or more anatomical structures of interest to a user; designating or defining the target position, either by choosing the position represented by some other image in the set or by defining an ideal anatomical position from a desired outcome, having the most relevant anatomical position, and designating the source image containing relevant information to be correlated to the target position; applying contouring, either manual or automatic, to the one or more medical image set, where the one or more medical image set includes a minimum set of common structures, where the structures include: organs, radiosensitive structures, skeletal structures, and skeletal muscles; generating a common coordinate system between the source and target using an auto-registration algorithm or a manual registration or a hybrid, whereby the user may set constraints on the registration in order to focus the registration on a structure of particular importance and then allow an algorithm to perform some limited registration; assigning each structure to one of three anatomical groups: high fidelity structure (HFS) for structures which are very easily delineated on the image sets and which have clearly defined allowable motions and deformations, medium fidelity structure (MFS) for structures which are easily delineated, have well known positional limits relative to some HFS but may have potential motion and deformation capability which may be more difficult to define, and low fidelity structures which may be difficult to delineate on imaging and whose potential motions and deformations may be difficult to define; assigning an anatomy specific transformation model to each structure where the transformation models (TM) include: data regarding the allowable motion of the specified structure inside the human body, tolerances for translation, rotation, and deformation per structures, assigning anatomy specific relative motion constraints (RMC) for each structure where the RMC contains data on the allowable motion of one structure relative to an adjacent structure; generating a deformation map consisting of an array of transformations per pixel defined first for the HFS group of structures where the motion and deformation, constrained by the TM, are defined which would take each structure from its orientation in the source image to its orientation in the target image relative to the common coordinate system, second for the MFS group where the motion and deformation, constrained by the assigned TM and RMC, are defined which would take each structure from its orientation in the source image to its orientation in the target position relative to the common coordinate system, and similarly for the LFS group; generating a source image, deformed by the animation process, by arranging voxels according to the transformations in the deformation map; applying smoothing and resampling as may be necessary or convenient for the application and use of the correlated source image data.

### BRIEF DESCRIPTION OF THE DRAWINGS

For the purposes of promoting an understanding of the principles of the present disclosure, reference is now made to the embodiments illustrated in the drawings, which are described below. The embodiments disclosed herein are not intended to be exhaustive or limit the present disclosure to the precise form disclosed in the following detailed description. Rather, the embodiments are chosen and described so that others skilled in the art can utilize their teachings. Therefore, no limitation of the scope of the present disclosure is thereby intended.

FIG. 1 is an embodiment of a system for medical imaging.

FIG. 2 is a schematic drawing for an embodiment of the disclosed system for medical imaging using anatomy guided image animation workflow.

FIG. 3 is a schematic drawing for an embodiment of the disclosed system for medical imaging using anatomy guided image animation workflow.

FIG. 4 is a flow chart for an embodiment of the disclosed method for medical imaging.

### DETAILED DESCRIPTION

In one or more embodiments, the system and method for medical imaging can include, but is not limited to: one or more computed tomography scanners, one or more magnetic resonance imaging scanners, one or more computers, one or more software modules, one or more processors, one or more transmitters, one or more receivers, one or more display units, one or more network interfaces, one or more detectors, one or more printers, and combinations thereof.

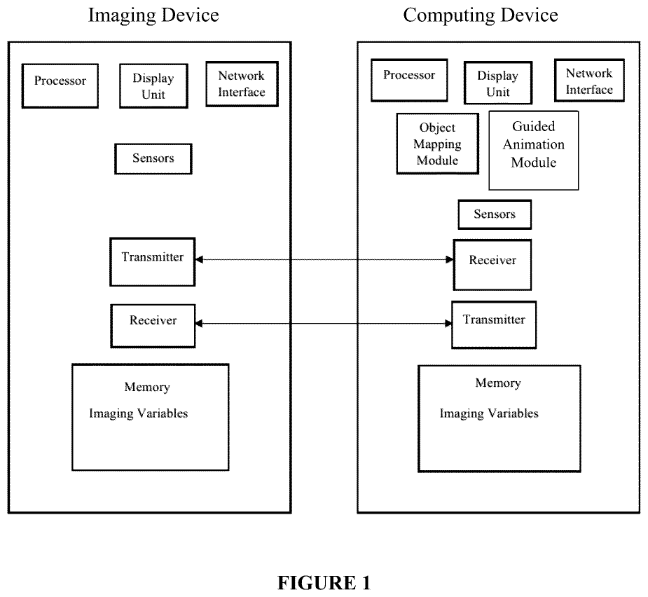

FIG. 1 shows an embodiment of a system for medical imaging disclosed herein. The system comprises a least an imaging device and a computer or other computing device. The imaging device may be computed tomography (CT), magnetic resonance (MR), positron emission tomography (PET), ultrasound (US), or any other form of imaging of an anatomy currently known in the art or later developed to achieve equivalent results. While the imaging devices presented herein largely relate to those used in medical treatment of humans, a person having ordinary skill will recognize that the same system and method disclosed herein can have broader applications to the veterinary sciences and other sciences requiring anatomical imaging. As seen in FIG. 1, the imaging device comprises at least a processor, a display unit, a network interface, one or more sensors, a transmitter in communication with the receiver of the computing device, a receiver in communication with the transmitter of the computing device, and a memory comprising one or more imaging variables. The disclosed computing device comprises a processor, a display unit, a network interface, an object mapping module, a guided deformable registration module, one or more sensors, a transmitter in communication with the receiver of the imaging device, a receiver in communication with the transmitter of the imaging device, and a memory comprising one or more imaging variables.

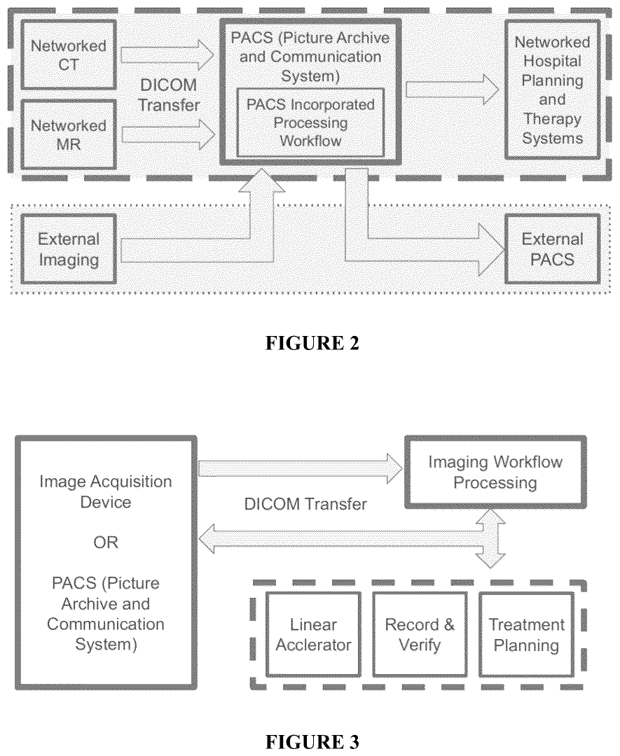

FIGS. 2 and 3 show schematic drawings for two embodiments of the guided deformable registration module of the system for medical imaging using anatomy guided image animation workflow. As shown in FIG. 1, the imaging device transmitter sends the images received by the imaging device to the receiver of the computing device, and those images are communicated to the guided deformable registration module. In one embodiment, as shown in FIG. 2, the image is sent using the Digital Imaging and Communications in Medicine (DICOM) standard, but those having skill in the art will recognize that other transmission methods and standards may be used to achieve equivalent results. Other external imaging may also be transmitted as seen in FIG. 2. The image is received by the Picture Archive and Communications System (PACS) of the guided deformable registration module, and the PACS comprises the image processing workflow (also known as the guided animation module) incorporated into the PACS. After processing the image through the workflow, the image may be sent to networked hospital planning and therapy systems, external PACS, or other locations desired by the user. The image may also be shown on the display unit of the computing device.

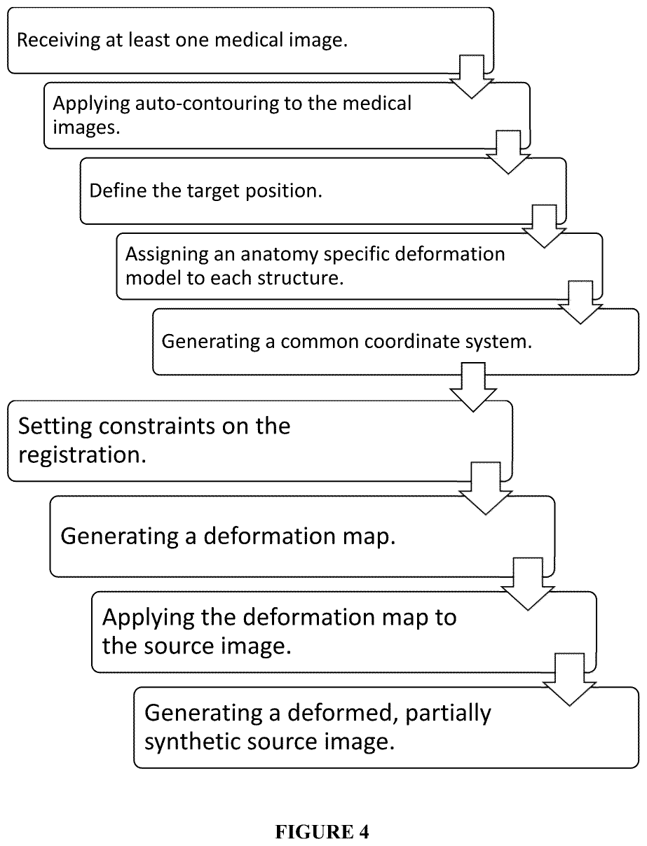

In the additional embodiment shown in FIG. 3, the image processing workflow is separate from the PACS, and the images may be received into the image processing workflow by either the imaging device (identified as the imaging acquisition devices in the drawings) or from the PACS via the DICOM standard, but those having skill in the art will recognize that other transmission methods and standards may be used to achieve equivalent results. In further embodiment shown in FIG. 3, the image processing workflow is in communication with a linear accelerator module, a record & verify module, and a treatment planning module. The process undertaken by the image processing workflow may be seen in FIG. 4.

The system and method for medical imaging requires the distinction of one volumetric image set as the source and the definition of a target position. The target is the ideal or desired position of the imaged patient. The target position may, in some embodiments, correspond to a volumetric image set which has the target patient position and anatomical situation most relevant to the final application of the correlated information. In radiation therapy, the target would be the position of the patient in the volumetric image acquired with the patient in the treatment position, or an abstract anticipated treatment position whether imaged or defined apart from imaging. The source is the volumetric image containing information required for the development of the treatment plan which may not be in the treatment position. The system and method for medical imaging describes a workflow for animating a source image for the purpose of generating a transformed source image containing the necessary information in a spatial arrangement corresponding to the target position.

The system and method for medical imaging utilizes a registration algorithm to create a common coordinate system between the source and target. The system and method for medical imaging utilizes a contouring algorithm to group pixels and/or voxels to generate delineated structures corresponding to skeletal structures, skeletal muscles, organs, anatomical features, and anatomical regions, and other categories or groups as needed. The system and method for medical imaging can use these groups of pixels and/or voxels as anatomical structures with known anatomical motions for each of the specific structures. In some embodiments, the system and method for medical imaging can perform computations on a per structure basis, reducing the required computations necessary for a given transformation from a simple count of the pixels and/or voxels in an image set, which can number in the millions or tens of millions of pixels and/or voxels, down to a few hundred computations. The system and method for medical imaging can use less computing power and less computing time than current methods. The system and method for medical imaging can yield higher accuracy and precision of the relative motions and a reduction in the number of calculations required to produce a deformed, partially synthetic source image that geometrically aligns with a target position.

The system and method for medical imaging can use groups of pixels and/or voxels as anatomical structures with known allowed anatomical motions for each of the specific structures. The anatomical structures can be used with known motions to generate a transformation map which defines the necessary motion to bring a given structure from its orientation in the source image to its orientation in the target image relative to the common coordinate system. In some embodiments, the system and method for medical imaging can group structures according to the ease with which the motion of the structure can be defined. High fidelity structures (HFS) being the most easily delineated structures with the most easily defined motions, medium fidelity structures (MFS) being relatively easy to delineate, but perhaps having a wider range of motions and deformations, and lower fidelity structures (LFS) having the least definable borders on imaging and the least definable allowed motions and deformations. For example, larger skeletal bones are easily visualized and delineated on CT, the allowable motion for these structures is relatively easy to define and could be defined as HFS. The orientation of a given larger skeletal bone being known will inform the possible orientations of numerous other anatomical structures nearby, e.g. skeletal muscles. A given skeletal muscle could then be defined as MFS. Further, lymph nodes in the groin and upper thorax are often of interest in radiation therapy, but their location is not trivial to define, and the position of the nodes can significantly vary with hip and leg position, or shoulder and arm position, respectively. Lymph nodes may be defined as LFS. In some embodiments, the system and method for medical imaging can use the transformations from HFS to inform constraints on MFS and thus to LFS, allowing greater accuracy and precision with the stepwise process. Pixels and voxels are thus not treated as individual intensity carriers, but as part of a whole structure. In some embodiments, the structures could be defined individually rather than the HFS, MFS, LFS categories, or according to other categories as may be appropriate to facilitate the animation.

The system and method for medical imaging can provide reliable dynamic articulation via an image animation workflow for medical imaging. The system and method for medical imaging can use known skeletal and/or muscular positions as anchor points adjusted to known orientations, either pre-defined or individually customized, to generate a transformation map by inferring tolerances for the motions of the structures that are either more difficult to delineate or whose motions are more difficult to define.

FIG. 4 shows an embodiment of a method for medical imaging. The method for medical imaging can include receiving one or more user inputs, one or more raw images, or a combination thereof. In some embodiments, the raw images can include computed tomography scans, magnetic resonance scans, positron emission tomography scans, ultrasound scans, and combinations thereof. In some embodiments, the method for medical imaging receives input of two or more 3D image sets. In some embodiments, the inputs could be two sets of the same modality, MR, CT, PET, or they could be 3D sets of differing modalities, commonly CT and MR, or CT and PET CT. Additional inputs can be optionally supplied by the user to adjust structural constraints-anatomical structures, which may include but is not limited to: radiology notes, medical correspondence between physicians, and manual inputs. In some embodiments, the system and method of medical imaging can include optical character recognition (OCR) technology can be included in the workflows. For example, the optical character recognition can allow for system and method for medical imaging to recognize and extract text or data from the images. In some embodiments, data can be captured from images and documents which have been scanned or digital uploaded. In some embodiments, artificial intelligence (AI) can be used to train the models and algorithms used in the workflow with information extracted from imaging, text, or other inputs.

In some embodiments and applications, e.g. as in radiation therapy, the workflow can be performed on a 3D source image set, acquired in advance of the radiation therapy, e.g. a PET/CT scan, and used to animate the 3D image set to align with an abstract ideal anticipated treatment position in preparation for the radiation therapy treatment planning. The animated 3D image set, having a new, ideal treatment position, can then be used to generate predictive graphical depictions of dose distributions superimposed on the source image which has been animated and transformed to the target (anticipated treatment) position. In some embodiments, AI may be used to generate the predictive dose distributions on the transformed source image through the image animation workflow. This workflow may be performed in an iterative fashion in some embodiments, in order to animate the source image to find, perfect, and set the anticipated treatment position in conjunction with the predictive dose distributions. In some embodiments, the predictive dose distributions can be used by radiation oncology physicians and professionals to set graphically guided dose-volume constraints on the treatment planning process. In some embodiments, this will allow the radiation oncologist to perform all of the physician tasks in one sitting as early as the consult or simulation, eliminating the time-consuming iterative review process.

In some embodiments, the workflow can be incorporated into the imaging process in order to assess needed changes in patient position. In radiation therapy, for example, when performing image-guided-radiation-therapy (IGRT) the workflow could be applied to 3D radiographic imaging by superimposing the contours from the treatment planning images of certain contoured structures of interest, skeletal structures, lungs, or other visualized structures, on the daily 3D images. The image guided animation workflow can utilize the known motion constraints to define 3 dimensional transformations of individual structures needed to bring the patient position into better agreement with the ideal anticipated target position. For example, from a 3D image of the pelvis, a deviation in the position of the inguinal lymph nodes could be observed and the position of the lower limbs could be assessed from the contours of the femoral heads. Anatomical motions could be defined to bring the anatomy into alignment with the ideal target position. Positional differences can be correlated with known 3D motions, congruent with the real, anatomical motion of the femur and the related motions of the lymph nodes, which can be subsequently defined and performed on the patient in order to bring the femur and related anatomy into alignment with the target position.

In some embodiments, the workflow can be incorporated into the imaging process in order to assess needed changes in patient position. In radiation therapy, for example, when performing image-guided-radiation-therapy (IGRT) the workflow could be applied to 2D radiographic imaging by superimposing the projection of contours from the treatment planning images of certain contoured structures of interest, skeletal structures, lungs, or other visualized structures, on the 2D images. The image guided animation workflow can utilize the known motion constraints to define 3 dimensional transformations of individual structures needed to bring the patient position into better agreement with the ideal anticipated target position. For example, from a 2D image of the shoulder and upper arm, the position of the humerus can be evaluated. Positional differences can be correlated with known 3D motions, congruent with the real, anatomical motion of the humerus, which can be subsequently defined and performed on the patient in order to bring the humerus and related anatomy into alignment with the target position.

In some embodiments, the workflow could be used to generate practical or likely anatomical motions, which may vary from the ideal target position, for the purpose of superimposing predictive dose distributions and subsequently generating a library of customized potential plan revisions. Utilizing the known anatomical motions of the structures of interest, the likelihood of such motions and their likely magnitudes, the user could identify likely anatomical motions which excessively deviate from the original treatment plan, per stated tolerances. Predictive plan revisions can be generated proactively allowing the rapid selection of an appropriate plan revision, during the course of radiation therapy, when the anatomical motions are observed outside the tolerances of the original plan.

In some embodiments, the workflow could be used to compare structures or positions between image sets in order to assess temporal changes such as disease progression and regression. A skilled user will see that the workflow could also be used to assess other temporal changes between image sets as may be relevant and appropriate in other applications and uses of anatomical imaging.

The system and method for medical imaging can include applying auto-contouring of a predefined set of anatomical structures including a minimum set of common structures. For example, the structure sets can include large organs and radiosensitive structures with the addition of skeletal structures and skeletal muscle as the starting place for developing the coarse level transformation map. Each contoured structure is assigned an anatomy specific deformation model. In some embodiments, the model can include known data regarding the allowable motion of the specified structure inside the human body. In some embodiments, the model can include models generated using artificial intelligence. In some embodiments, the model can include attributes for translation, rotation, and deformation per anatomical structure. In some embodiments, the model can include tolerances for translation, rotation, relative motions between structures, and deformation per structure. In some embodiments, the method for medical imaging can permit translational and rotational motion as well as deformations of anatomical structures, like the lungs and diaphragm, while for other anatomical structures, like bones, little or no deformations. For example, the model can include constraints on the motion of one vertebral body relative to the one adjacent to it while keeping the overall shape of the bone.

The method for medical imaging can generate a common coordinate system for the inputs and/or raw images. In some embodiments, the method for medical imaging can generate an anchor point for a rigid registration between the inputs and/or raw images. In some embodiments, method for medical imaging can include an auto-registration algorithm. In some embodiments, method for medical imaging can include a manual registration or a hybrid registration, whereby the user can set constraints on the registration in order to focus the registration on a structure of particular importance and then allow software modules to perform some limited registration.

In some embodiments, the system and method for medical imaging can generate a displacement map. The displacement map can include the displacement of structures from the image set to the anatomical structure using the common coordinate system. In some embodiments, the user can provide input into the coarse structure deformation map to generate a new deformation map with the user input. The system and method for medical imaging can generate a deformation map using a registration module. In some embodiments, system and method for medical imaging can examine the borders of contoured structures, contrasting in each image set. In some embodiments, the system and method for medical imaging can use interpolation and/or smoothing between anatomical structures to make a final deformation map. The method for medical imaging can apply the final deformation map to the source image set and/or raw image set to align the geometry of the anatomical structure to the target image set. The method for medical imaging can generate a deformed, partially synthetic source image which geometrically aligns with the target position.

In some embodiments, the software modules for system and method for medical imaging can include routines, modules, logic blocks, and other symbolic representations of operations on data within one or more electronic devices. These routines, modules, and logic blocks can provide a self-consistent sequence of steps or instructions leading to a desired result, such as anatomy guided image animation workflow. In some embodiments, the physical manipulations can be provided by electric or magnetic signals capable of being stored, transferred, compared, and otherwise manipulated by a computer. These signals can include data, bits, values, elements, symbols, characters, terms, numbers, and/or strings. In some embodiments, data can be represented as physical (e.g., electronic) quantities within the computer's logic circuits, registers, and/or memories, and is transformed into other data similarly represented as physical quantities within the electronic device.

In some embodiments, the system and method for medical imaging can include one or more artificial intelligence modules for processing and analyzing the medical images. For example, the artificial intelligence modules can include, but is not limited to, machine learning modules, deep learning modules, and combinations thereof. In another example, the system and method for medical imaging a can include, but is not limited to, an artificial neural network (ANN), multilayered artificial neural network, recurrent neural network (RNN), convolution neural network (CNN), and combinations thereof. In some embodiments, the system and method for medical imaging can include logic, if-then rules, decision trees, and combinations thereof. In some embodiments, an artificial intelligence module can include, but is not limited to, abstruse statistical techniques that enable the system and method to improve at a task with experience. In some embodiments, an artificial intelligence module can include algorithms that allow the software to train itself to perform tasks, such as the anatomy guided image animation workflow.

In some embodiments, the system and method of medical imaging can include, but is not limited to: training artificial intelligence models to recognize and categorize objects within images and can be applied to include anatomy recognition, object detection, and image categorization. The artificial intelligence module can be trained by minimizing a loss function or cost function. For example, the method is based on the idea of optimizing the network's parameters to minimize the difference, or empirical risk, between the predicted output and the actual target values in a given dataset. Gradient based methods such as backpropagation can be used to estimate the parameters of the network. During the training phase, the artificial intelligence model can learn from labeled training data by iteratively updating their parameters to minimize a defined loss function or cost function.

The artificial intelligence training can include identifying, learning, and generating logic rules to store, manipulate, and apply to the image. The artificial intelligence module can allow for the adaptation of the system to better handle a task, such as anatomy guided image animation workflow, by learning from training image set samples. The artificial intelligence module can include adjusting the weights and/or thresholds of cost functions or loss functions to improve the accuracy of the results, which can be done by minimizing the observed errors. The artificial intelligence learning can complete when examining additional observations does not usefully reduce the error rate. As long as its output error continues to decline, artificial intelligence learning continues. Artificial intelligence learning attempts to reduce the total of the differences across the observations. In some embodiments, the artificial intelligence learning models can include optimization theory and statistical estimation.

The system and method of medical imaging can include training the artificial intelligence model to learn to recognize patterns and features in the images that are associated with the provided labels. In some embodiments, the system and method of medical imaging can include, but is not limited to collecting a dataset of medical images. In some embodiments, the system and method of medical imaging can include generating an artificial intelligence model to identify anatomical features patterns in the images collected in the data set. In some embodiments, the system and method of medical imaging can include, but is not limited: dividing an image into segments to analyze specific regions independently; and utilizing artificial intelligence algorithms to improve the quality of images by reducing noise, adjusting brightness and contrast, and enhancing sharpness. In some embodiments, the system and method for medical imaging can classify the images based on content, layout, or structure.

The artificial intelligence module of the system and method for medical imaging can include one or more steps. For example, the artificial intelligence module can include, but is not limited to: image recognition, image classification, image segmentation, image enhancement, data collection, pre-processing, image artifact recognition, model generating, model training, feature extraction, validation, inference, learning, post-processing, image generation, and combination thereof.

In some embodiments, the system and method for medical imaging for image processing can begin with collecting a large dataset of labeled images relevant to the task, such as anatomical structure recognition or image classification. The processed images and data can then be easily converted into structured data making it easier for them to be searched, stored, and sorted for a more efficient document management process.

In some embodiments, the system and method for image processing can include preprocessing the medical images. The preprocessing of the medical images can include resizing, normalization, and combinations thereof to ensure consistency and improve model performance.

In some embodiments, the raw medical images or preprocessed medical images can be input into the artificial intelligence model for training. The artificial intelligence model analyzes the features of the input image and produces predictions or outputs based on its training. In one or more embodiments, the artificial intelligence model can automatically learn and extract features from images. For example, the artificial intelligence model can detect patterns like edges, textures, deformation of anatomical features.

In some embodiments, during training, the model adjusts its internal weights and biases based on the differences between its predictions and the actual values in the training data. In some embodiments, optimization algorithms can be used to iteratively update the model's parameters to minimize prediction errors. In some embodiments, a separate validation dataset can be used to monitor the model's performance during training and prevent overfitting, e.g., when the model memorizes training data but performs poorly on new data. Once trained, the artificial intelligence model can make inferences about new, unseen medical images to make predictions about anatomical delineation or deformation. The processed images or outputs can be visualized or further utilized in various applications, such as medical diagnosis.

The artificial intelligence module can include, but is not limited to, supervised learning or unsupervised learning. In unsupervised learning, the artificial intelligence module can analyze medical images and data and finds patterns and makes predictions without any other guidance. In supervised learning, the artificial intelligence module can use guidance from a user to label or annotate the input data. In some embodiment, the artificial intelligence module can include classification, where the module learns to predict the category in which the data belongs, and regression, where the module deduces a numeric cost function based on the medical images.

In some embodiments, the system and method for medical imaging can include annotating training data associated with a set of medical images for the learning of the artificial intelligence module. In some embodiments, artificial intelligence can annotate or re-annotate a training image set. In some embodiments, annotation of a training image set can be manually performed by a user.

After the artificial intelligence model is trained, it should be able to identify the important features in new, unanalyzed images. For example, new images can be introduced to the trained model and using the previously learned patterns, should be able to recognize patterns and features in the images. In some embodiments, the classification of data can be based on previously acquired knowledge or statistical data extrapolated from patterns and/or their representation. In some embodiments, the artificial intelligence module can be used in pattern recognition to identify the artifacts in an image.

In some embodiments, the system and method for medical imaging can include generating a dataset of synthetic images to monitor the models' performance in recognizing features. In some embodiments, the system and method for medical imaging and workflows can be optimized with automation to improve the accuracy and precisions of image analysis. Once the fully trained model is ready and deployed, it can be improved with cycles of retraining with new data to finetune the models' performance based on user feedback.

The system and method for image processing can include one or more post-processing techniques on the medical images. For example, the post-processing the medical images can include, but is not limited to: image restoration, image morphological processing, image augmentation, and combination thereof. For example, the system and method can include a variety of digital coloring techniques, such as HSI Hue-Saturation-Intensity (HIS), CMY (Cyan-Magenta-Yellow), RGB (Red-Green-Blue), and combinations thereof. In another example, the post-processing the medical images can include image compression, image decompression, and combination thereof. In some embodiments, the post-processing can enable adjustments to image resolution and size, whether for image reduction or restoration depending on the situation, without lowering image quality below a desirable level. In some embodiments, the post-processing can include image file compression, morphological processing, segmentation, and combinations thereof. In some embodiments, the post-processing can include the Scale-invariant Feature Transform (SIFT), the Speeded Up Robust Features (SURF), and the PCA (Principal Component Analysis). In some embodiments, the post-processing can divide an image into segments, and each segment can be processed further by a computer.

In some embodiments, the post-processing can include zooming, blurring, sharpening, converting from grayscale to color, identifying edges and vice versa, image retrieval, and image recognition are included. Recovering lost resolution and reducing noise are the goals of image restoration techniques. Either the frequency domain or the image domain can be used for image processing techniques. Image post-processing can be employed to enhance an image's quality, remove unwanted artefacts from an image, or create a new image.

In some embodiments, the system and method for medical imaging can generate multiple image animations and related data that can be used to give an anatomical history over time for a given patient. Aggregated patient data can be used to develop key predictors in disease progression, improve accuracy of staging and other prognostic indicators used to select a course of therapy, and flag the need to consider a change in therapy over the long term. Specifically, more information over time related to nodal progression, and other areas as may arise.

In some embodiments, the system and method for medical imaging can correlate body surface contours or positioning cone-beam CT images with simulation CT positioning to determine if mobile structures are appropriately localized for treatment. In some embodiments, the system and method for medical imaging can include real-time adaptive radiotherapy.

The system and method for medical imaging can include a computer readable storage device and can include instructions that, in response to execution, cause a system including a processor to perform operations, including annotating training data associated with a set of images for anatomical feature learning. The processor also performs operations, including incrementally updating an artificial intelligence model for an engineering component based on the feature learning process. The processor also performs operations, including providing a display device to display information associated with the system and method for medical imaging in a human-interpretable format, such as deformed, partially synthetic source image, where the deformed, partially synthetic source image geometrically aligns with the target image.

In some embodiments, the system and method for medical imaging can include the generation of structural motion and position variables, including the relative dependence between structures. In some embodiments, the system and method for medical imaging can include entering known structural motion and the known relative dependence between structures. For example, known limitations on the relative motion of structures can be defined, along with a chain of dependence. For example, a motion of structure 3 depends on the motion of structure 2 which depends on the motion of structure 1, so their locations can be inferred one from the other.

The generation of structural motion and position variables can begin with a rigid registration between two image sets. The necessary structures can be contoured. The focus on the interdependence of structures, rather than pixel intensity, necessitates the user or algorithm choosing an easily delineated structure as the anchor structure, and setting an anchor point, (x,y,z), in the centroid of that structure, which can be the origin in both image sets. Given that structure 1 and structure 2 can be localized on both image sets, they can be contoured, and their locations compared between the sets. For example, the origin can be denoted point A on both sets, the centroid of structure 1, thus Aₛ=Aₜ=(0, 0, 0). The centroid of structure 2, denoted point B, can be defined on both the source, Bₛ, and target image set, B₁, relative to the centroid of structure 1. Point Bₛ can be expressed, Bₛ=(xₛ, yₛ, zₛ), and similarly with Bₜ=(xₜ, yₜ, zₜ). The motion between the source and target, can then be defined as the difference between the two, ΔB=(Δx_{B}, Δy_{B}, Δz_{B}), where Δx_{B}=xₛ−xₜ, Δy_{B}=yₛ−yₜ, Δz_{B}=zₛ−zₜ.

Suppose structure 3 is easily localized on the source image but is not visible on the target. Further, the motion of structure 3 depends on the motion of structure 2, according to some known anatomical motions, defined as function ƒ(x,y,z), The centroid of structure 3 is denoted point C=(xₛ, yₛ, zₛ) on the source image. The motion of the centroid of structure 3, denoted point C, can be expressed ΔC=(Δx_{c}, Δy_{c}, Δz_{c})=ΔB+ƒ(x,y,z), one only needs to know the location of structure 2, the value of ΔB, and the definition of ƒ(x,y,z), in order to generate a region of probable location for structure 3. Then, in three dimensions, the location of the centroid of structure 3 can be analytically predicted. Further functions, g(x,y,z), and h(x,y,z), can be defined to correct for known anatomical rotations and deformations.

Rotational motion in three dimensions g(x,y,z) and a deformation function, h(x,y,z), can be defined to account for stretching, shrinking, growing, etc. In the case that structure 3 is of particular interest and is easily localizable on the source image set but not easily localizable on the target image set, the known relation between structures 2 and 3 can be used to provide a probable localization volume.

The translational motions, ΔC, the rotational motions, g(x,y,z), and a deformation function, h(x,y,z), will define relative motions for structure 3 from the source image to the target. The translational motion defines changes in position parallel to each axis. The rotational motions define rotations around each axis in the reference frame in order to correct the orientation of the structure from the source image to the target. The deformation function defines changes in morphology, stretching, shrinking, etc.

The system and method for medical imaging can locate on the target image, a structure of interest which is localized on the source image. Sometimes the target may not contain enough information for the localization of the structure of interest.

By definition, the anchor structure needs no translational corrections, however for the anchor structure in the source image motion changes, functions g and h, will be added to bring the position and orientation into agreement with the target. The deviations assigned to the source can be combined with the parameters for the target in order to generate new parameters for the source. Translations can be 0 as the centroids have been defined to be identical in location, but rotational motion and deformation may be non-zero. A parameter string can be used to store motion parameters for a given structure in the source image and can be written, [Δx, Δy, Δz, g(x), g(y), g(z), h(x), h(y), h(z)]. A skilled user can understand and devise many ways to store the motion in a string or array or other computational instrument as may suit the needs of a particular application.

Structures are evaluated beginning with the anchor structure and proceeding with structures in order of dependence. For example, L2 is a vertebral body whose position has a mutual dependence on the position of L1, and similarly, T12. In some embodiments, this generation of variables can proceed with L2 by first looking at the centroid of the structure in the source image and the centroid in the target position.

The above-described features and applications can be implemented as software processes that are specified as a set of instructions recorded on a computer readable storage medium (also referred to as computer readable medium). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections. Some implementations include electronic components, for example microprocessors, storage and memory that store computer program instructions in a machine-readable or computer-readable medium (alternatively referred to as computer-readable storage media, machine-readable media, or machine-readable storage media, any or all of which can be non-transitory). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic or solid state hard drives, read-only and recordable BLU-RAY® discs, ultra density optical discs, any other optical or magnetic media, and floppy disks. The computer-readable media can store a computer program that is executable by at least one processing unit and includes sets of instructions for performing various operations. Examples of computer programs or computer code include machine code, for example is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, for example application specific integrated circuits (ASICs) or field programmable gate arrays (FPGAs). In some implementations, such integrated circuits execute instructions that are stored on the circuit itself.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

In this specification, the term “software” is meant to include firmware residing in read-only memory or applications stored in magnetic storage or flash storage, for example, a solid-state drive, which can be read into memory for processing by a processor. Also, in some implementations, multiple software technologies can be implemented as sub-parts of a larger program while remaining distinct software technologies. In some implementations, multiple software technologies can also be implemented as separate programs. Finally, any combination of separate programs that together implement a software technology described here is within the scope of the subject technology. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

These functions described above can be implemented in digital electronic circuitry, in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

The subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some aspects of the disclosed subject matter, a server transmits data (e.g., an HTML page) to a client device (e.g., for purposes of displaying data to and receiving user input from a user interacting with the client device). Data generated at the client device (e.g., a result of the user interaction) can be received from the client device at the server.

One of ordinary skill in the art will readily appreciate that alternate but functionally equivalent components, materials, designs, and equipment may be used. The inclusion of additional elements may be deemed readily apparent and obvious to one of ordinary skill in the art. Specific elements disclosed herein are not to be interpreted as limiting, but rather as a basis for the claims and as a representative basis for teaching one of ordinary skill in the art to employ the present invention.

Various terms have been defined above. To the extent a term used in a claim is not defined above, it should be given the broadest definition persons in the pertinent art have given that term as reflected in at least one printed publication or issued patent.

Certain embodiments and features have been described using a set of numerical upper limits and a set of numerical lower limits. It should be appreciated that ranges including the combination of any two values, e.g., the combination of any lower value with any upper value, the combination of any two lower values, and/or the combination of any two upper values are contemplated unless otherwise indicated. It should also be appreciated that the numerical limits may be the values from the examples. Certain lower limits, upper limits and ranges appear in at least one claims below. All numerical values are “about” or “approximately” the indicated value, and consider experimental error and variations that would be expected by a person having ordinary skill in the art.

It is understood that any specific order or hierarchy of steps in the processes disclosed is an illustration of example approaches. Based upon design preferences, it is understood that the specific order or hierarchy of steps in the processes may be rearranged, or that all illustrated steps be performed. Some of the steps may be performed simultaneously. For example, in certain circumstances, multitasking and parallel processing may be advantageous. Moreover, the separation of various system components illustrated above should not be understood as requiring such separation, and it should be understood that the described program components and systems can generally be integrated together in a single software product or packaged into multiple software products.

Various modifications to these aspects will be readily apparent, and the generic principles defined herein may be applied to other aspects. Thus, the claims are not intended to be limited to the aspects shown herein, but is to be accorded the full scope consistent with the language claims, where reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject technology.

As used herein, the singular forms “a”, “an”, and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. As used herein, use of the term “including” as well as other forms, such as “includes,” and “included,” is not limiting.

All patents, patent applications, provisional applications, and publications referred to or cited herein are incorporated by reference in their entirety, including all figures and tables, to the extent they are not inconsistent with the explicit teachings of this specification.

What is claimed is:

1. A system for medical imaging, comprising:
an imaging device, comprising:
a transmitter
a receiver; and
a memory, comprising one or more imaging variables;
a computing device, comprising:
a processor;
a display unit;
a network interface;
an object mapping module;
a guided animation module;
one or more sensors;
a receiver;
a transmitter; and
a memory, comprising one or more imaging variables;
a picture archive communication system; and
an image processing workflow;
wherein the transmitter of the imaging device is in communication with the receiver of the computing device;
wherein the transmitter of the computing device is in communication with the receiver of the computing device;
wherein the image processing workflow comprises:
an input;
an output;
one or more constraints;
one or more deformation models; and
functionality to perform the following actions:
auto-contouring, manual contouring, or a hybrid of a medical image received by the input;
defining a target position;
assigning the one or more deformation models to a structure;
generating common coordinate systems;
generating one or more deformation maps;
setting constraints on a registration;
applying one or more deformation maps to the medical image; and
generate a deformed, partially synthetic source image.

2. The system of claim 1, wherein the imaging device further comprises:
a processor;
a display unit;
a network interface; and
one or more sensors.

3. The system of claim 1, wherein the image processing workflow is stored within the picture archive and communications system.

4. The system of claim 1, further comprising:
an external imaging input;
an external picture archive and communication system output; and
networked planning and therapy systems.

5. The system of claim 1, further comprising:
a linear accelerator module;
a record & verify module; and
a treatment planning module.

6. A method of medical imaging, wherein the method comprises:
(a) providing a system, comprising:
a computing device, comprising a processor;
a picture archive and communications system; and
an image processing workflow, comprising:
an input;
an output;
one or more constraints;
one or more deformation models; and
computer coding and hardware configured to perform one or more steps of the method;
(b) receiving one or more medical images, wherein the medical images each comprise one or more anatomical structures;
(c) auto-contouring, manual contouring, or a hybrid to the one or more medical images;
(d) generating a deformation map,
(e) assigning an anatomy specific deformation model to each structure;
(f) defining an ideal target position;
(g) generating a common coordinate system with an anchor point for a rigid registration between the one or more medical images and the ideal target position;
(h) applying the one or more constraints to rigid registrations;
(i) performing step-wise registration between the one or more medical images and the ideal target position;
(j) generating a first deformation map using the generated common coordinate system, comprising a showing of a displacement of structures between the one or more medical images and the ideal target position;
(k) generating a second deformation map using a registration algorithm;
(l) applying the second deformation map to the one or more medical images received to align the one or more medical images to a target position; and
(m) generating a deformed, partially synthetic source image, wherein the deformed, partially synthetic source image geometrically aligns with the target position.

7. The method of claim 6, wherein the medical image comprises one or more voxels, one or more pixels, or a combination thereof.

8. The method of claim 6, wherein the medical image comprises a computed tomography scan.

9. The method of claim 6, wherein the medical image comprises a magnetic resonance image.

10. The method of claim 6, wherein the at least one medical image set comprises a minimum set of common structures, and wherein the structures comprise one or more of the following: organs, radiosensitive structures, skeletal structures, and skeletal muscles, and any other structures which are relevant and appropriate to the purpose.

11. The method of claim 6, wherein the deformation model comprises one or more of the following data points: the allowable motion of the specified structure inside the human body, tolerances for translation, rotation, relative motion between structures, and deformation per structure, constraints on the motion of one structure, either absolute or relative to an adjacent structure, and any other relevant information useful to the deformation model.

12. The method of claim 7, wherein the idea target position comprises either a position of a patient in one of the medical images or an abstract ideal position being fixed or dynamic.

13. The method of claim 6, where in the registration algorithm:
(a) examines one or more borders of the structures identified and a contrast in the one or more medical images; and
(b) performs interpolation and smoothing between one or more structures applied to the second deformation map.

14. A non-transitory computer readable medium comprising instruction which, when implemented by one or more computers, causes the one or more computers to:
(a) receiving one or more medical images, wherein the medical image is a computed tomography scan or a magnetic resonance image, wherein the medical image comprises one or more anatomical structures of interest to a user, wherein the medical image comprises: one or more voxels, one or more pixels, or a combination thereof;
(b) applying auto-contouring, manual contouring, or a hybrid to the one or more medical images to generate a deformation map, wherein the one or more medical image set comprises a minimum set of common structures, wherein the structures comprise: organs, radiosensitive structures, skeletal structures, and skeletal muscles, and any other structures which are relevant and appropriate to the purpose;
(c) assigning an anatomy specific deformation model to each structure, wherein the deformation models comprise: data regarding the allowable motion of the specified structure inside the human body, tolerances for translation, rotation, relative motion between structures, and deformation per structure, constraints on the motion of one structure, either absolute or relative to an adjacent structure, and any other relevant information useful to the model;
(d) defining an ideal target position being either the position of the patient in one of the medical images, or an abstract ideal position being fixed or dynamic, or an ideal position defined in another way;
(e) generating a common coordinate system with an anchor point for a rigid registration between the one or more images and the ideal target position;
(f) using an auto-registration algorithm or a manual registration or a hybrid, whereby the user may or may not set constraints on the registration in order to focus the registration on a structure of particular importance and then allow an algorithm to perform some step-wise registration between the one or more medical images and the ideal target position starting with easily delineated structures with well-known tolerances, e.g. skeletal structures, and moving toward structures with characteristics that are not as easily defined;
(g) generating a deformation map using the common coordinate system, wherein the first deformation map shows the displacement of structures between the one or more medical images and the ideal target position;
(h) generating a deformation map using a registration algorithm examining the borders of the contoured structures and the contrast in each image set, interpolation and smoothing between structures being applied to the deformation map, and examining any other relevant and appropriate features of the one or more medical images as may be useful to the purpose;
(i) applying the deformation map to the source image set to align the geometry of the source to a target position; and
generating a deformed, partially synthetic source image, wherein the deformed, partially synthetic source image geometrically aligns with the target position.

15. The non-transitory computer readable medium of claim 14, wherein the processing modules configured to transform a raw image into an output image, wherein module is configured to implement an image transformation operation using a trained artificial intelligence model.
